(19) United States
(12) Patent Application Publication
(10) Pub. No.: US 2004/0098106 A1
(43) Pub. Date: May 20, 2004

(54) Intraluminal prostheses and carbon dioxide-assisted methods of impregnating same with pharmacological agents

(76) Inventors: Michael S. Williams, Santa Rosa, CA (US); Joseph M. DeSimone, Chapel Hill, NC (US)

Correspondence Address: MYERS BIGEL SIBLEY & SAJOVEC, PO BOX 37428, RALEIGH, NC, 27627, US

(21) Appl. No.: 10/662,757

(22) Filed: Sep. 15, 2003

Related U.S. Application Data
(60) Provisional application No. 60/426,125, filed on Nov. 14, 2002.

(51) Int. Cl. A61F 2/06; A61L 2/00

(52) U.S. Cl. 623/1.15; 427/2.24; 623/1.42; 623/1.46

(57) ABSTRACT

Intraluminal prostheses and methods of impregnating same with pharmacological agents for delivery within a body of a subject are provided. An intraluminal prosthesis comprising polymeric material is immersed in a mixture of carrier fluid and pharmacological agent(s). The mixture of carrier fluid and pharmacological agent is pressurized for a time sufficient to cause the polymeric material of the intraluminal prosthesis to swell such that the carrier fluid and pharmacological agent at least partially penetrate the swollen polymeric material. Pressure is then removed such that the carrier fluid diffuses out of the swollen polymeric material and such that a predetermined amount of the pharmacological agent remains elutably trapped within the polymeric material.

### RELATED APPLICATION

This application claims the benefit of U.S. Provisional Application No. 60/426,125, filed Nov. 14, 2002, the disclosure of which is incorporated herein by reference in its entirety as if set forth fully herein.

### FIELD OF THE INVENTION

The present invention relates generally to impregnating polymeric materials and, more particularly, to methods of impregnating polymeric materials with pharmacological agents.

### BACKGROUND OF THE INVENTION

Stents are typically used as adjuncts to percutaneous transluminal balloon angioplasty procedures, in the treatment of occluded or partially occluded arteries and other blood vessels. As an example of a balloon angioplasty procedure, a guiding catheter or sheath is percutaneously introduced into the cardiovascular system of a patient through the femoral arteries and advanced through the vasculature until the distal end of the guiding catheter is positioned at a point proximal to the lesion site. A guidewire and a dilatation catheter having a balloon on the distal end are introduced through the guiding catheter with the guidewire sliding within the dilatation catheter. The guidewire is first advanced out of the guiding catheter into the patient's vasculature and is directed across the arterial lesion. The dilatation catheter is subsequently advanced over the previously advanced guidewire until the dilatation balloon is properly positioned across the arterial lesion. Once in position across the lesion, the expandable balloon is inflated to a predetermined size with a radiopaque liquid at relatively high pressure to radially compress the atherosclerotic plaque of the lesion against the inside of the artery wall and thereby dilate the lumen of the artery. The balloon is then deflated to a small profile so that the dilatation catheter can be withdrawn from the patient's vasculature and blood flow resumed through the dilated artery.

Balloon angioplasty sometimes results in short or long term failure (restenosis). That is, vessels may abruptly close shortly after the procedure or restenosis may occur gradually over a period of months thereafter. To counter restenosis following angioplasty, implantable intraluminal prostheses, commonly referred to as stents, are used to achieve long term vessel patency. A stent functions as scaffolding to structurally support the vessel wall and thereby maintain luminal patency, and are transported to a lesion site by means of a delivery catheter.

Types of stents may include balloon expandable stents, spring-like, self-expandable stents, and thermally expandable stents. Balloon expandable stents are delivered by a dilitation catheter and are plastically deformed by an expandable member, such as an inflation balloon, from a small initial diameter to a larger expanded diameter. Self-expanding stents are formed as spring elements which are radially compressible about a delivery catheter. A compressed self-expanding stent is typically held in the compressed state by a delivery sheath. Upon delivery to a lesion site, the delivery sheath is retracted allowing the stent to expand. Thermally expandable stents are formed from shape memory alloys which have the ability to expand from a small initial diameter to a second larger diameter upon the application of heat to the alloy.

It may be desirable to provide localized pharmacological treatment of a vessel at the site being supported by a stent. Thus, sometimes it is desirable to utilize a stent both as a support for a lumen wall as a well as a delivery vehicle for one or more pharmacological agents. Unfortunately, the metallic materials typically employed in conventional stents are not generally capable of carrying and releasing pharmacological agents. Previously devised solutions to this dilemma have been to join drug-carrying polymers to metallic stents. Additionally, methods have been disclosed wherein the metallic structure of a stent has been formed or treated so as to create a porous surface that enhances the ability to retain applied pharmacological agents. However, these methods have generally failed to provide a quick, easy and inexpensive way of loading drugs onto intraluminal prostheses, such as stents. Moreover, it would be desirable to replace toxic organic solvents and plasticizers conventionally used to impregnate polymeric materials with pharmacological agents with more environmentally benign alternatives.

### SUMMARY OF THE INVENTION

Methods of impregnating intraluminal prostheses with pharmacological agents for delivery within a body of a subject are provided. According to embodiments of the present invention, an intraluminal prosthesis (e.g., a stent, drug delivery device, etc.) formed from polymeric material, or having a coating of polymeric material, is immersed in a mixture of carrier fluid and pharmacological agent(s). The mixture is pressurized (e.g., via pressurized carbon dioxide) for a time sufficient to cause the polymeric material to swell and such that the carrier fluid and pharmacological agent(s) can at least partially penetrate the swollen polymeric material. The pressure is then removed (completely or partially) such that the carrier fluid diffuses out of the swollen polymeric material and such that a predetermined amount of the pharmacological agent(s) remains elutably trapped within the polymeric material.

According to embodiments of the present invention, a method of impregnating an intraluminal prosthesis with pharmacological agent(s) includes placing an intraluminal prosthesis formed from polymeric material, or having a coating of polymeric material, within a pressure vessel. The interior of the pressure vessel is pressurized to a predetermined pressure (e.g., via pressurized carbon dioxide). A mixture of a carrier fluid and pharmacological agent(s) is supplied into the pressure vessel and is exposed to the polymeric material for a time sufficient to swell the polymeric material such that the carrier fluid and pharmacological agent(s) at least partially penetrate the swollen polymeric material. The pressure in the pressure vessel is then released (completely or partially) such that the carrier fluid diffuses out of the swollen polymeric material and such that a predetermined amount of the pharmacological agent(s) remains elutably trapped within the polymeric material.

According to embodiments of the present invention, carbon dioxide can be utilized to alter the diffusion coefficients of various pharmacological agent-polymer matrices by modifying polymer permeability.

According to embodiments of the present invention, a method of impregnating an intraluminal prosthesis with a pharmacological agent includes exposing polymeric material of an intraluminal prosthesis to carbon dioxide under conditions sufficient to tackify the polymeric material. A pharmacological agent is applied in micronized, dry form to the tackified polymeric material. A membrane layer is then applied to the intraluminal prosthesis, and is configured to allow the pharmacological agent to elute therethrough when the intraluminal prosthesis is deployed within a body of a subject.

According to embodiments of the present invention, a method of impregnating an intraluminal prosthesis with multiple pharmacological agents includes exposing polymeric material of an intraluminal prosthesis to carbon dioxide under conditions sufficient to tackify multiple portions of the polymeric material. A respective different pharmacological agent is applied in micronized, dry form to each respective tackified portion of the polymeric material. A membrane layer is then applied to the intraluminal prosthesis, and is configured to allow the pharmacological agents to elute therethrough when the intraluminal prosthesis is deployed within a body of a subject.

According to embodiments of the present invention, a method of impregnating an intraluminal prosthesis with multiple pharmacological agents includes exposing polymeric material of an intraluminal prosthesis to carbon dioxide under conditions sufficient to tackify a portion of the polymeric material. A first pharmacological agent is applied in micronized, dry form to the tackified portion of the polymeric material. A first membrane layer is applied to the intraluminal prosthesis, and is configured to allow the first pharmacological agent to elute therethrough when the intraluminal prosthesis is deployed within a body of a subject. A second pharmacological agent is applied to the first membrane layer. A second membrane layer is then applied to the intraluminal prosthesis such that the second pharmacological agent is sandwiched between the first and second membrane layers. The second membrane layer is configured to allow the second pharmacological agent to elute therethrough when the intraluminal prosthesis is deployed within a body of a subject.

According to embodiments of the present invention, an intraluminal prosthesis includes a tubular body portion comprising polymeric material, one or more pharmacological agents in dry, micronized form attached directly to the tubular body portion, and a membrane attached to the tubular body portion and overlying the one or more pharmacological agents. The membrane is configured to allow the one or more pharmacological agents to elute therethrough when the intraluminal prosthesis is deployed within a body of a subject.

According to embodiments of the present invention, carbon dioxide can be used to facilitate the loading the polymeric material of intraluminal prostheses with radiopaque materials, such as, but not limited to, bismuth trioxide or barium sulfate. For example, the polymeric material can be subjected to pressurized carbon dioxide for a time sufficient to cause the polymeric material to swell and such that radiopaque material can at least partially penetrate the swollen polymeric material. As would be understood by those skilled in the art, radiopaque materials can facilitate monitoring the placement of an intraluminal prosthesis, such as a stent, within a subject via known radiography techniques.

Embodiments of the present invention are particularly advantageous because the use of carbon dioxide precludes the need for heat which can cause degradation and/or denaturization of pharmacological agents loaded into intraluminal prostheses.

### BRIEF DESCRIPTION OF THE DRAWINGS

FIGS. 1-2 are flowcharts of operations for impregnating polymeric material with pharmacological agents, according to embodiments of the present invention.

FIG. 3 is a flowchart of operations for applying pharmacological agents to polymeric material, according to embodiments of the present invention.

FIG. 4 is a perspective view of an intraluminal prosthesis produced in accordance with embodiments of the present invention.

FIG. 5 is a cross-sectional view of the intraluminal prosthesis of FIG. 4 taken along lines 5-5.

FIG. 6 is a cross-sectional view of the intraluminal prosthesis of FIG. 4 with an second pharmacological agent and a second membrane, according to embodiments of the present invention.

### DETAILED DESCRIPTION OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The term “eluting” is used herein to mean the release of a pharmacological agent from a polymeric material. Eluting may also refer to the release of a material from a substrate via diffusional mechanisms or by release from a polymeric material/substrate as a result of the breakdown or erosion of the material/substrate.

The term “erodible” as used herein refers to the ability of a material to maintain its structural integrity for a desired period of time, and thereafter gradually undergo any of numerous processes whereby the material substantially loses tensile strength and mass. Examples of such processes comprise enzymatic and non-enzymatic hydrolysis, oxidation, enzymatically-assisted oxidation, and others, thus including bioresorption, dissolution, and mechanical degradation upon interaction with a physiological environment into components that the patient's tissue can absorb, metabolize, respire, and/or excrete. The terms “erodible” and “degradable” are intended to be used herein interchangeably.

The term “dosage regimen” is used herein to describe both exogenously administered and internally administered pharmacological agents. A dosage regimen includes both an amount of a pharmacological agent and timers) that each dose is to be taken. A dosage regimen may also indicate whether a pharmacological agent is to be taken with food or not, and whether other pharmacological agents are to be avoided.

The term “everolimus” is used herein to mean any member of the macrolide family of pharmacological agents.

The term “hydrophobic” is used herein to mean not soluble in water.

The term “hydrophilic” is used herein to mean soluble in water.

The term “lumen” is used herein to mean any inner open space or cavity of a body passageway.

The terms “polymer” and “polymeric material” are synonymous and are to be broadly construed to include, but not be limited to, homopolymers, copolymers, terpolymers, and the like.

The term “prosthesis” is used herein in a broad sense to denote any type of intraluminal prosthesis or other device which is implanted in the body of a subject for some therapeutic reason or purpose including, but not limited to stents, drug delivery devices, etc.

The term “subject” is used herein to describe both human beings and animals (e.g., mammalian subjects) for medical, veterinary, testing and/or screening purposes.

As used herein, phrases such as “between X and Y” and “between about X and Y” should be interpreted to include X and Y.

As used herein, phrases such as “between about X and Y” mean “between about X and about Y.”

As used herein, phrases such as “from about X to Y” mean “from about X to about Y.”

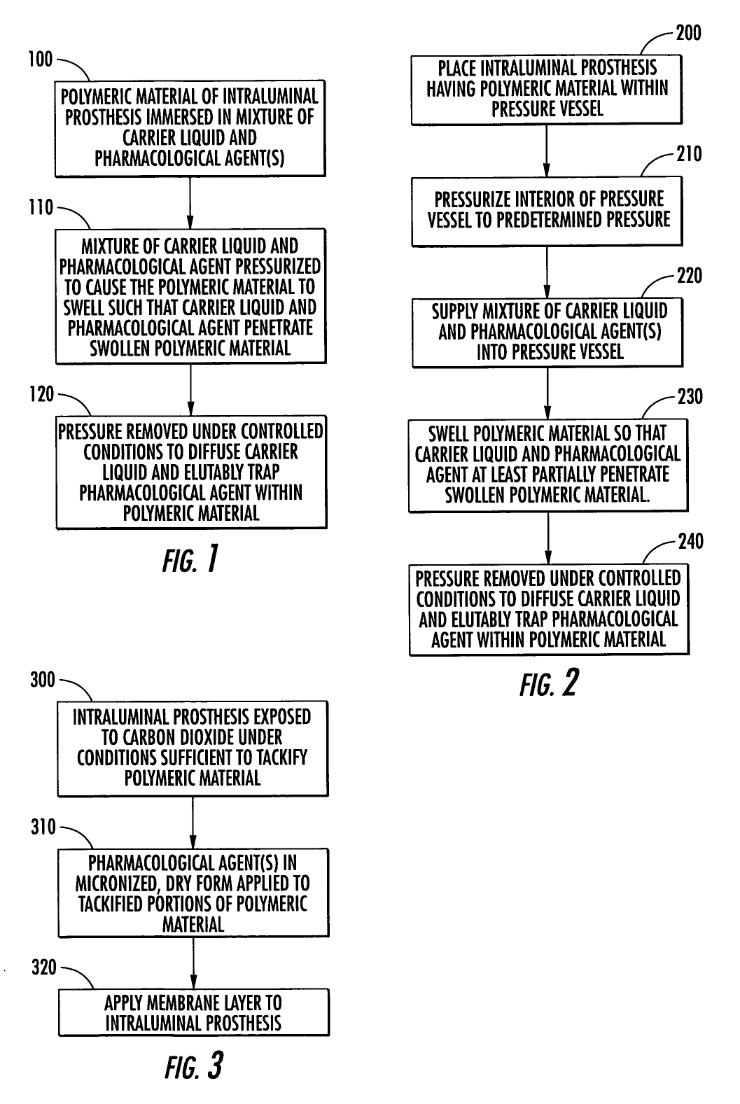

Referring now to FIGS. 1-3, methods of impregnating polymeric material of intraluminal prostheses (e.g., stents, etc.) with pharmacological agents for delivery within a body of a subject, according to embodiments of the present invention are illustrated. Embodiments of the present invention can be employed in conjunction with a number of manufacturing processes associated with producing intraluminal prostheses including, but not limited to, extrusion, pultrusion, injection molding, compression molding, etc. Moreover, embodiments of the present invention may be utilized in batch, semicontinuous, or continuous processes.

Referring initially to FIG. 1, an intraluminal prosthesis (e.g., a stent, drug delivery device, etc.) comprising polymeric material (e.g., formed from polymeric material, or having a coating of polymeric material) is immersed in a mixture of carrier fluid and pharmacological agent(s) (Block 100). According to embodiments of the present invention, one or more pharmacological agents may be infused within polymeric material of an intraluminal prosthesis or within a polymeric coating surrounding an intraluminal prosthesis.

The carrier fluid may be a gas, liquid, or supercritical fluid. The carrier fluid may be heterogeneous or homogeneous in composition, i.e., may be a single phase composition or contain one or more additional phases, such as in the form of a microemulsion, emulsion, dispersion, suspension, etc. The carrier fluid may comprise, consist of, or consist essentially of carbon dioxide. Where multiple phases are found in the carrier fluid, carbon dioxide may be the continuous phase. One or more other ingredients may be included in the carrier fluid, such as co-solvents (i.e., water or organic co-solvents such as ethanol and methanol), surfactants or the like may be included. Where one or more organic co-solvents are included, it or they may be polar or nonpolar (or at least one of each). Where one or more surfactants are included, it or they may comprise a carbon dioxide-philic group coupled to either a lipophilic (hydrophobic) or hydrophilic group, a conventional surfactant comprising a liphophilic (hydrophobic) group coupled to a hydrophilic group, or one or more of each. The carrier fluid may comprise at least 30, 40, 50, 60, 70, 80 or 90 percent by weight of carbon dioxide. When water is present in the carrier fluid, the water may comprise from about 0.01, 0.1, or 0.5 to about 1, 5, 10 or 20 percent by weight of the composition, or more.

In general, pharmacological agents suitable for inclusion in prosthesis materials and/or coatings, according to embodiments of the present invention include, but are not limited to, drugs and other biologically active materials, and may be intended to perform a variety of functions, including, but not limited to: anti-cancer treatment (e.g., Resan), anti-clotting or anti-platelet formation, the prevention of smooth muscle cell growth, migration, proliferation within a vessel wall. Pharmacological agents may include antineoplastics, antimitotics, antiinflammatories, antiplatelets, anticoagulants, antifibrins, antithrombins, antiproliferatives, antibiotics, antioxidants, and antiallergic substances as well as combinations thereof. Examples of antineoplastics and/or antimitotics include paclitaxel (cytostatic and ant-inflammatory) and it's analogs and all compounds in the TAXOL® (Bristol-Myers Squibb Co., Stamford, Conn.) family of pharmaceuticals, docetaxel (e.g., TAXOTERE® from Aventis S. A., Frankfurt, Germany) methotrexate, azathioprine, vincristine, vinblastine, fluorouracil, doxorubicin hydrochloride (e.g., ADRIAMYCIN® from Pharmacia & Upjohn, Peapack N.J.), and mitomycin (e.g., MUTAMYCIN® from Bristol-Myers Squibb Co., Stamford, Conn.). Examples of antiinflammatories include Sirolimus and it's analogs (including but not limited to Everolimus and all compounds in the Limus family of pharmaceuticals), glucocorticoids such as dexamethasone, methylprednisolone, hydrocortisone and betamethasone and non-steroidal antiinflammatories such as aspirin, indomethacin and ibuprofen. Examples of antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.) Examples of cytostatic or antiproliferative agents or proliferation inhibitors include everolimus, actinomycin D, as well as derivatives and analogs thereof (manufactured by Sigma-Aldrich, Milwaukee, Wis.; or COSMEGEN® available from Merck & Co., Inc., Whitehouse Station, N.J.), angiopeptin, angiotensin converting enzyme inhibitors such as captopril (e.g., CAPOTEN® and CAPOZIDE® from Bristol-Myers Squibb Co., Stamford, Conn.), cilazapril or lisinopril (e.g., Prinivilo and PRINZIDE® from Merck & Co., Inc., Whitehouse Station, N.J.); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name MEVACOR® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include alphainterferon, genetically engineered epithelial cells, and dexamethasone.

U.S. Pat. No. 4,994,033 to Shockey et al.; U.S. Pat. No. 5,674,192 to Sahatian et al. and U.S. Pat. No. 5,545,208 to Wolff et al. disclose catheters comprising absorbable/biodegradable polymers or hydrogels containing the desired dosage of a drug. Stents incorporating drug delivery may be found, for example, in U.S. Pat. No. 5,766,710 to Turnlund et al.; U.S. Pat. No. 5,769,883 to Buscemi et al.; U.S. Pat. No. 5,605,696 to Eury et al.; U.S. Pat. No. 5,500,013 to Buscemi et al.; U.S. Pat. No. 5,551,954 to Buscemi et al. and U.S. Pat. No. 5,443,458 to Eury, each of which is incorporated herein by reference in its entirety.

Pharmacological agents, according to embodiments of the present invention, may be hydrophilic or hydrophobic. For hydrophilic pharmacological agents, the carrier fluid may be water. For hydrophobic pharmacological agents, the carrier fluid may be a supercritical fluid, such as liquid carbon dioxide. An exemplary hydrophobic pharmacological agent according to embodiments of the present invention is everolimus. Everolimus is a proliferation inhibitor that targets primary causes of chronic rejection in organ transplantation patients and may also be effective for the prevention of restenosis.

According to embodiments of the present invention, carbon dioxide may be employed as a fluid in a liquid, gaseous, or supercritical phase. If liquid carbon dioxide is used, the temperature employed during the process is typically below 31° C. If gaseous carbon dioxide is used, the phase may be employed at high pressure. As used herein, the term “high pressure” generally refers to carbon dioxide having a pressure from about 50 to about 500 bar. Carbon dioxide may be utilized in a “supercritical” phase. As used herein, “supercritical” means that a fluid medium is above its critical temperature and pressure, i.e., about 31° C. and about 71 bar for carbon dioxide. The thermodynamic properties of carbon dioxide are reported in Hyatt, J. Org. Chem. 49: 5097-5101 (1984).

Typically, supercritical fluids are gases at ambient temperature and pressure. However, when maintained at or above its critical point, a supercritical fluid displays properties of both a gas and a liquid. In particular, a supercritical fluid has the solvent characteristics of a liquid, but the low surface tension of a gas. Accordingly, as with a gas, a supercritical fluid can more readily diffuse into polymeric material. While any of a variety of supercritical fluids may be utilized in accordance with embodiments of the present invention, carbon dioxide is a particularly desirable supercritical fluid because it is substantially non-reactive and nontoxic (i.e., inert).

Carbon dioxide is non-toxic, non-flammable, chemically inert, completely recoverable, abundant and inexpensive. Carbon dioxide has properties that are between those of many liquids and gases. At room temperature and above its vapor pressure, carbon dioxide exists as a liquid with a density comparable to organic solvents but with excellent wetting properties and a very low viscosity. Above its critical temperature and pressure (31° C. and 73.8 bar), carbon dioxide is in the supercritical state and has gas-like viscosities and liquid-like densities. Small changes in temperature or pressure cause dramatic changes in the density, viscosity, and dielectric properties of supercritical carbon dioxide, making it an unusually tunable, versatile, and selective solvent.

Still referring to FIG. 1, the mixture of carrier fluid and pharmacological agent is pressurized for a time sufficient to cause the polymeric material of the intraluminal prosthesis to swell such that the carrier fluid and pharmacological agent at least partially penetrate the swollen polymeric material (Block 110). According to embodiments of the present invention, pressure can be added by the use of pressurized carbon dioxide, or by the use of a different second pressurized gas. A different second pressurized gas, such as one or more inert gases, may be helium, nitrogen, argon, etc., or combinations thereof.

For pharmacological agents soluble in carbon dioxide (e.g., hydrophobic agents), carbon dioxide may be utilized as both the carrier fluid and the pressurizing medium. For pharmacological agents not soluble in carbon dioxide (e.g., hydrophilic agents), the pharmacological agent and carrier fluid may be pressurized by an overlying blanket of carbon dioxide. Carbon dioxide is well known to those skilled in the art to be capable of swelling and plasticizing polymeric materials. Carbon dioxide is capable of partitioning into polymeric materials that are in its presence. When this occurs it can dramatically lower the glass transition temperature of the amorphous phase of the polymer. When this occurs, the diffusivity of a third component can increase dramatically. Such plasticization can enable the partitioning of third components, like a pharmaceutical agent, into the material. Conventionally, heat is required to increase glass transition temperature. Unfortunately, heating can be difficult with pharmaceutical agents that are thermally labile.

According to embodiments of the present invention, a carrier fluid such as carbon dioxide can be utilized to alter the diffusion coefficients of various pharmacological agent-polymer matrices by modifying permeability of the polymeric material.

Pressure is then removed such that the carrier fluid diffuses out of the swollen polymeric material and such that a predetermined amount of the pharmacological agent remains elutably trapped within the polymeric material (Block 120). The term “elutably trapped” means that the pharmacological agent is disposed within the polymeric material in such a way that it can elute (at a predetermined rate) therefrom when the intraluminal prosthesis is deployed within the body of a subject. The step of removing pressure is carried out under controlled conditions after a predetermined time and according to a predetermined schedule to insure that the desired predetermined amount of the pharmacological agent remains. Controlled conditions include controlling one or more of the following parameters in a predetermined pattern: temperature, rate of temperature change, pressure, rate of pressure change, carrier fluid quantity, concentration of the pharmacological agent in the carrier fluid, concentration of cosolvents and surfactants etc. These parameters can control the concentration of the pharmacological agent entrapped within the polymeric material after depressurization has been achieved. Moreover, as these parameters are varied, concentration gradients of the pharmacological agent entrapped within the polymeric material after depressurization can be achieved. Such concentration gradients can give rise to modified elution profiles of the pharmacological agent.

According to embodiments of the present invention, the polymeric material of an intraluminal prosthesis may be erodible (or the intraluminal prosthesis may have a erodible coating). Exemplary erodible materials that may be utilized in accordance with embodiments of the present invention include, but are not limited to, surgical gut, silk, cotton, liposomes, poly(hydroxybutyrate), polycarbonates, polyacrylates, polyanhydrides, polyethylene glycol, poly(ortho esters), poly(phosphoesters), polyesters, polyamides (such as polyamides derived from D-glucose), polyphosphazenes, poly(p-dioxane), poly(amino acid), polyglactin, and copolymers thereof, erodible hydrogels, natural polymers such as collagen and chitosan, etc. See, e.g., U.S. Pat. No. 5,723,508 to Healy et al. Particular examples of suitable erodible polymers include, but are not limited to, aliphatic polyester polymers such as poly(lactic acid), poly(L-lactic acid), poly(D,L-lactic acid), poly(glycolic acid), poly(D-lactic-co-glycolic acid), poly(L-lactic-co-glycolic acid), poly (D,L-lactic-co-glycolic acid), poly(ε-caprolactone), poly(valerolactone), poly(hydroxy butyrate) (inlcuding poly(hydroxy butyrate valerate)), poly(hydrovalerate), polydioxanone, poly(propylene fumarate), etc., including copolymers thereof such as polylactic acid-polyethylene glycol block copolymer, and poly(ethyleneoxide)-poly(butylenetetraphthalate), poly(lactic acid-co-lysine), poly(ε-caprolactone copolymers), poly(L-lactic acid copolymers), etc. See, e.g., J. Oh et al., PCT Application WO 99/59548 at page 2. Additional examples of erodible polymers are set forth in U.S. Pat. No. 5,916,585 to Cook et al. at col. 9 line 53 to col. 10 line 22. The molecular weight (that is, average molecular weight) of the polymer may be from 1,000, 10,000, 100,000 or 500,000 to 2,000,000 or 4,000,000 Daltons, or more.

According to embodiments of the present invention, an intraluminal prosthesis may be composed of polymeric material that is not erodible. Exemplary non-erodible materials include, but are not limited to, fluoropolymers, polyesters, PET, polyethylenes, polypropylenes, etc., and/or ceramics, such as hydroxyapetite.

Referring now to FIG. 2, a method of impregnating an intraluminal prosthesis with a pharmacological agent, according to other embodiments of the present invention, is illustrated. An intraluminal prosthesis (e.g., a stent, drug delivery device, etc.) comprising polymeric material (e.g., formed from polymeric material, or having a coating of polymeric material) is placed within a pressure vessel (Block 200). The interior of the pressure vessel is pressurized to a predetermined pressure via a pressurizing media (e.g., carbon dioxide) (Block 210). A mixture of carrier fluid and pharmacological agent(s) is supplied into the pressure vessel (Block 220) and is forced into contact with the polymeric material of the intraluminal device for a time sufficient to swell the polymeric material so that the carrier fluid and pharmacological agent at least partially penetrate the swollen polymeric material (Block 230). Selected portions of the polymeric material may be masked so as to create portions or regions of the polymeric material having different concentrations of the pharmacological agent entrapped in it, or to partition one pharmacological agent in one region of the prosthesis from another pharmacological agent in a second (or third or fourth) region of the prosthesis. The mask can be a protective layer of a material that is plasticized to a lesser extent, perhaps not plasticized at all, rendering the partitioning of the pharmacological agent in the areas not protected by the mask to be higher than in the areas protected by the mask. Any of a variety of masking techniques can be employed to achieve a selective tackifying pattern.

Pressure is then released from the pressure vessel such that the carrier fluid (e.g., carbon dioxide) diffuses out of the swollen polymeric material and such that a predetermined amount of the pharmacological agent remains elutably trapped within the polymeric material (Block 240). Removal of the carrier fluid from the polymeric material may be facilitated by any suitable means, including pumping and/or venting from the pressure vessel, as would be understood by one skilled in the art.

Referring now to FIG. 3, a method of impregnating an intraluminal prosthesis with a pharmacological agent, according to other embodiments of the present invention, is illustrated. An intraluminal prosthesis (e.g., a stent, drug delivery device, etc.) comprising polymeric material (e.g., formed from polymeric material, or having a coating of polymeric material) has the polymeric material (or portions thereof) exposed to carbon dioxide under conditions sufficient to tackify the polymeric material (Block 300). The term “tackify” means that the surface of a polymeric material is exhibiting adhesive properties (e.g., has become “sticky”) such that micronized particles can be adhesively secured thereto. The particles can also be fluidized or dispersed, with or without the aid of additives like surfactants, in the carbon dioxide medium to facilitate the even distribution of the pharmacological agent adhered to the polymeric material. Selected portions of the polymeric material may be masked so as to selectively tackify portions of the polymeric material. The mask can be a protective layer of a material that is plasticized to a lesser extent, perhaps not plasticized at all, rendering the adhesion of particles to the areas not protected by the mask. Any of a variety of masking techniques can be employed to achieve a selective tackifying pattern.

One or more pharmacological agents in micronized, dry form are applied directly to the tackified portions of the polymeric material (Block 310). The one or more pharmacological agent(s) are attached directly to the body portion without the use of a separate or additional adhesive material. Layers of multiple pharmacological agents may be utilized with a lower-most layer being attached directly to the body portion.

The pharmacological agent(s) are supplied in the form of dry, micronized or sub-micronized particles that readily adhere to the tackified polymeric material. A variety of pharmacological agents are commercially available in such form having a particle size of about 1 to 0.05 microns. Examples of such pharmacological agents include but are not limited to antibiotics, anti-thrombotics, anti-restenotics, and antineoplastics.

A particularly desirable antineoplastic pharmacological agent in micronized, dry form is Paclitaxel. Paclitaxel is an antineoplastic that is used to treat various cancers including, but not limited to, cancer of the ovaries, breast, certain types of lung cancer, cancer of the skin and mucous membranes more commonly found in patients with acquired immunodeficiency syndrome (AIDS), etc.

Additionally, any such micronized or sub-micronized pharmacological agents can be combined in any of various combinations in order to dispense a desired cocktail of pharmacological agents. For example, a number of different pharmacological agents can be combined in each particle. Alternatively, micronized particles of individual pharmacological agents can be intermixed prior to application to the tackified polymeric material.

According to embodiments of the present invention, different pharmacological agents can be applied to different portions of an intraluminal prosthesis. Application of micronized or sub-micronized particles may be achieved by any of a number of well known methods. For example, the particles may be blown onto tackified polymeric material or tackified polymeric material may be rolled in a powder of micronized particles.

According to embodiments of the present invention, multiple pharmacological agents may be attached directly to an intraluminal prosthesis in layers.

One or more membrane layers may be applied to the intraluminal prosthesis after the application of micronized particles to tackified portions of the polymeric material (Block 320). A membrane layer is configured to allow pharmacological agent(s) to elute therethrough when the intraluminal prosthesis is deployed within a body of a subject. The membrane may allow the pharmacological agent to elute at a predetermined rate when the intraluminal prosthesis is deployed within a body of a subject.

According to embodiments of the present invention, multiple membranes may be layered within different types and/or amounts of pharmacological agents therebetween. The multiple layer configuration can allow the multiple pharmacological agents to elute in correlation with a disease process, thus targeting varied aspects of a disease in its progression.

According to embodiments of the present invention, the membrane layer may encapsulate all of the polymeric material of an intraluminal prosthesis. According to other embodiments, the membrane layer may encapsulate only selected portions of the polymeric material (e.g., only the tackified portions). Membrane layer material is selected for its biocompatibility as well as its permeability to a pharmacological agent. A membrane layer may also serve as an aid in deployment within a subject.

The chemical composition of the membrane layer and that of a pharmacological agent in combination with the thickness of the membrane layer will determine the diffusion rate of the pharmacological agent. Examples of suitable materials for a membrane layer according to embodiments of the present invention includes, but is not limited to, ethylene vinyl alcohol, ethylene vinyl acetate, polyethylene glycol, etc. Alternatively, fluorocarbon films may be employed to serve as a membrane layer according to embodiments of the present invention. According to embodiments of the present invention, membrane layer material may be erodible. According to embodiments of the present invention, membrane layer material may be the same material as the underlying prosthesis (or a similar material).

Embodiments of the present invention described above with respect to FIGS. 1-3 may be carried out using apparatus known to those skilled in the art. An exemplary apparatus for use in impregnating intraluminal prostheses with pharmacological agents according to the methods of FIGS. 1-2 is illustrated and described in U.S. Pat. No. 5,808,060 to Perman et al., which is incorporated herein by reference in its entirety.

Referring now to FIGS. 4-5, an intraluminal prosthesis 10, that may be produced according to embodiments of the present invention, is illustrated. The illustrated prosthesis 10 is a stent and includes a tubular body portion 12 having a first end 14, a second end 16, and a flow passage 18 defined therethrough from the first end 14 to the second end 16. The body portion 12 is sized for intraluminal placement within the vasculature of a subject and is expandable from a first, reduced cross-sectional dimension (i.e., contracted configuration) to a second enlarged cross-sectional dimension (i.e., expanded configuration) so that the body portion 12 can be transported intraluminally to a treatment site and then expanded to the second enlarged cross-sectional dimension so as to engage and support the vascular wall at the treatment site. The body portion 12 is formed at least in part from an erodible, polymeric material or a coating of erodible, polymeric material. The polymeric material may comprise polymers oriented uniaxially and/or biaxially. According to other embodiments, the body portion 12 may be formed at least in part from non erodible material.

According to embodiments of the present invention, one or more pharmacological agents (represented by cross-hatching 15) in dry, micronized form may be attached directly to the polymeric material 13 of the body portion 12, or to a polymeric coating surrounding the body portion 12, or portions thereof. In the illustrated embodiment, a membrane 20 is attached to the body portion 12 and overlies the one or more pharmacological agents 15. The membrane 20 is configured to allow the one or more pharmacological agents 15 to elute therethrough when the intraluminal prosthesis is deployed within a body of a subject.

If a plurality of pharmacological agents are utilized, the plurality of pharmacological agents may be homogeneously distributed on the body portion 12, or heterogeneously distributed on the body portion 12.

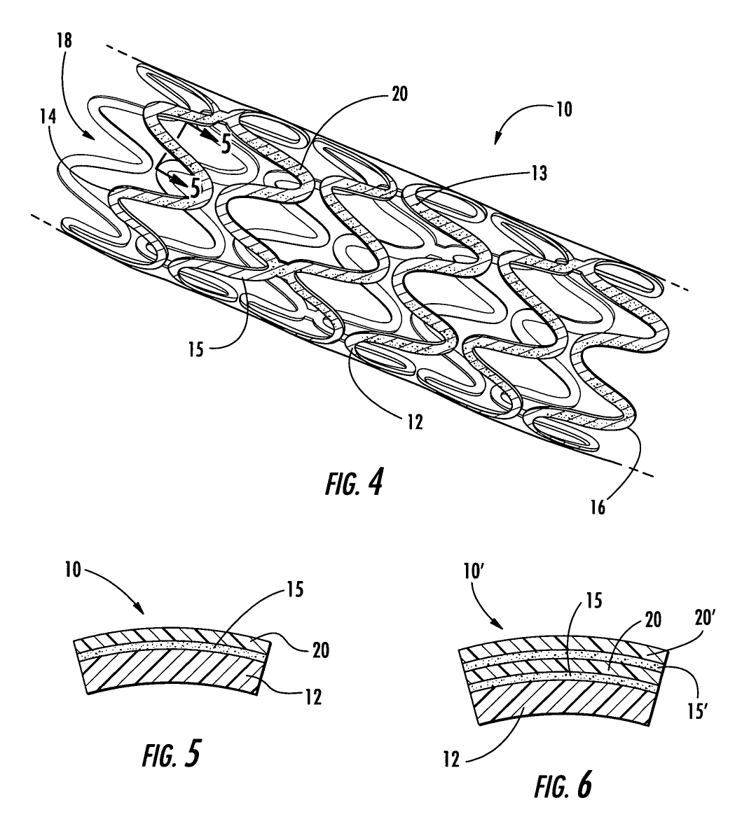

Referring to FIG. 6, an intraluminal prosthesis 10′, that may be produced according to embodiments of the present invention, is illustrated. The illustrated intraluminal prosthesis 10′ includes a first pharmacological agent 15 in micronized, dry form attached to the body portion 12 and a first membrane layer 20 overlying the first pharmacological agent 15 as described above with respect to FIGS. 4-5. The illustrated intraluminal prosthesis 10′, further includes a second pharmacological agent 15′ attached to the first membrane layer 20 and a second membrane layer 20′ overlying the second pharmacological agent 15′ such that the second pharmacological agent 15′ is sandwiched between the first and second membrane layers 20, 20′. The second membrane layer 20′ is configured to allow the second pharmacological agent 15′ to elute therethrough when the intraluminal prosthesis 10′ is deployed within a body of a subject. The illustrated intraluminal prosthesis 10′ thereby allows the sequential elution of the first and second pharmacological agents 15, 15′, preferably at predetermined and controlled rates.

Intraluminal prostheses provided in accordance with embodiments of the present invention may be employed in sites of the body other than the vasculature including, but not limited to, biliary tree, esophagus, bowels, tracheo-bronchial tree, urinary tract, etc.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed is:

1. A method of impregnating an intraluminal prosthesis with a pharmacological agent, comprising:
immersing an intraluminal prosthesis comprising polymeric material in a mixture of a carrier fluid and a pharmacological agent;
pressurizing the mixture of carrier fluid and pharmacological agent for a time sufficient to cause the polymeric material to swell such that the carrier fluid and pharmacological agent at least partially penetrate the swollen polymeric material; and
removing the pressure such that the carrier fluid diffuses out of the swollen polymeric material and such that a predetermined amount of the pharmacological agent remains elutably trapped within the polymeric material.

2. The method of claim 1, wherein the carrier fluid is carbon dioxide, and wherein the pharmacological agent is hydrophobic.

3. The method of claim 2, wherein the pharmacological agent comprises everolimus.

4. The method of claim 1, wherein the carrier fluid is water, and wherein the pharmacological agent is hydrophilic.

5. The method of claim 4, wherein pressurizing the mixture of carrier fluid and pharmacological agent comprises subjecting the mixture of carrier fluid and pharmacological agent to pressurized carbon dioxide.

6. The method of claim 2, wherein the carbon dioxide is present in a supercritical state.

7. The method of claim 6, wherein the carbon dioxide contains one or more of a co-solvent, a surfactant, and a co-surfactant.

8. The method of claim 1, wherein the carrier fluid is configured to alter diffusion coefficients of the polymeric material.

9. The method of claim 8, wherein the co-solvent is selected from the group consisting of ethanol and methanol.

10. The method of claim 1, wherein the intraluminal prosthesis is a stent.

11. The method of claim 1, wherein the polymeric material is erodible.

12. The method of claim 1, wherein the polymeric material is non-erodible.

13. The method of claim 1, wherein the polymeric material is a coating on a portion of the intraluminal prosthesis.

14. The method of claim 11, wherein the erodible polymeric material is selected from the group consisting of, surgical gut, silk, cotton, liposomes, poly(hydroxybutyrate), polycarbonate, polyacrylate, polyanhydride, polyethylene glycol, poly(ortho esters), poly(phosphoesters), polyesters, polyamides, polyphosphazenes, poly(p-dioxane), poly(amino acid), polyglactin, erodible hydrogels, collagen, chitosan, poly(lactic acid), poly(L-lactic acid), poly(D,L-lactic acid), poly(glycolic acid), poly(D-lactic-co-glycolic acid), poly(L-lactic-co-glycolic acid), poly (D,L-lactic-co-glycolic acid), poly(ε-caprolactone), poly(valerolactone), poly(hydroxy butyrate), poly(hydrovalerate), polydioxanone, poly(propylene fumarate), poly(ethyleneoxide)-poly(butylenetetraphthalate), poly(lactic acid-co-lysine), poly(L-lactic acid) and poly(ε-caprolactone) copolymers.

15. The method of claim 1, wherein the step of removing pressure is carried out under controlled conditions.

16. The method of claim 15, wherein the step of removing pressure is carried out under controlled conditions in which at least one parameter selected from the group consisting of temperature, rate of temperature change, pressure, rate of pressure change, carrier fluid quantity, and rate of carrier fluid quantity, is controlled in a predetermined pattern.

17. The method of claim 1, further comprising:
immersing the intraluminal prosthesis in a mixture of a carrier fluid and radiopaque material; and
pressurizing the mixture of carrier fluid and radiopaque material for a time sufficient to cause the polymeric material to swell such that the carrier fluid and radiopaque material at least partially penetrate the swollen polymeric material.

18. A method of impregnating an intraluminal prosthesis with a predetermined amount of a pharmacological agent, comprising:
immersing an intraluminal stent comprising erodible polymeric material in a mixture of carbon dioxide and pharmacological agent, wherein the erodible polymeric material is selected from the group consisting of, surgical gut, silk, cotton, liposomes, poly(hydroxybutyrate), polycarbonate, polyacrylate, polyanhydride, polyethylene glycol, poly(ortho esters), poly(phosphoesters), polyesters, polyamides, polyphosphazenes, poly(p-dioxane), poly(amino acid), polyglactin, erodible hydrogels, collagen, chitosan, poly(lactic acid), poly(L-lactic acid), poly(D,L-lactic acid), poly(glycolic acid), poly(D-lactic-co-glycolic acid), poly(L-lactic-co-glycolic acid), poly (D,L-lactic-co-glycolic acid), poly(ε-caprolactone), poly(valerolactone), poly(hydroxy butyrate), poly(hydrovalerate), polydioxanone, poly(propylene fumarate), poly(ethyleneoxide)-poly(butylenetetraphthalate), poly(lactic acid-co-lysine), poly(L-lactic acid) and poly(ε-caprolactone) copolymers;
pressurizing the mixture of carbon dioxide and pharmacological agent for a time sufficient to cause the polymeric material to swell such that the carbon dioxide and pharmacological agent at least partially penetrate the swollen polymeric material; and
removing the pressure such that the carbon dioxide diffuses out of the swollen polymeric material and such that a predetermined amount of the pharmacological agent remains elutably trapped within the polymeric material.

19. The method of claim 18, wherein the pharmacological agent is everolimus.

20. The method of claim 18, wherein the polymeric material is a coating on a portion of the intraluminal prosthesis.

21. The method of claim 18, wherein the carbon dioxide is present in a supercritical state.

22. The method of claim 18, wherein the carbon dioxide is configured to alter diffusion coefficients of the polymeric material.

23. The method of claim 18, wherein the intraluminal prosthesis is a stent.

24. A method of impregnating an intraluminal prosthesis with a pharmacological agent, comprising:
immersing an intraluminal prosthesis comprising erodible polymeric material in a mixture of water and a hydrophilic pharmacological agent, wherein the erodible polymeric material is selected from the group consisting of, surgical gut, silk, cotton, liposomes, poly(hydroxybutyrate), polycarbonate, polyacrylate, polyanhydride, polyethylene glycol, poly(ortho esters), poly(phosphoesters), polyesters, polyamides, polyphosphazenes, poly(p-dioxane), poly(amino acid), polyglactin, erodible hydrogels, collagen, chitosan, poly(lactic acid), poly(L-lactic acid), poly(D,L-lactic acid), poly(glycolic acid), poly(D-lactic-co-glycolic acid), poly(L-lactic-co-glycolic acid), poly (D,L-lactic-co-glycolic acid), poly(ε-caprolactone), poly(valerolactone), poly(hydroxy butyrate), poly(hydrovalerate), polydioxanone, poly(propylene fumarate), poly(ethyleneoxide)-poly(butylenetetraphthalate), poly(lactic acid-co-lysine), poly(L-lactic acid) and poly(ε-caprolactone) copolymers;
pressurizing the mixture of water and pharmacological agent with carbon dioxide for a time sufficient to cause the polymeric material to swell such that the water and pharmacological agent at least partially penetrate the swollen polymeric material; and
removing the pressure such that the water diffuses out of the swollen polymeric material and such that a predetermined amount of the pharmacological agent remains elutably trapped within the polymeric material.

25. The method of claim 24, wherein the polymeric material is a coating on a portion of the intraluminal stent.

26. The method of claim 24, wherein the carbon dioxide is present in a supercritical state.

27. The method of claim 26, wherein the carbon dioxide contains one or more of a co-solvent, a surfactant, and a co-surfactant.

28. The method of claim 27, wherein the co-solvent is selected from the group consisting of ethanol and methanol.

29. The method of claim 24, wherein the intraluminal prosthesis is a stent.

30. The method of claim 24, further comprising:
immersing the intraluminal prosthesis in a mixture of a carbon dioxide and radiopaque material; and
pressurizing the mixture of carbon dioxide and radiopaque material for a time sufficient to cause the polymeric material to swell such that the carbon dioxide and radiopaque material at least partially penetrate the swollen polymeric material.

31. A method of impregnating an intraluminal prosthesis with a pharmacological agent, comprising:
placing an intraluminal prosthesis within a pressure vessel, wherein a portion of the intraluminal prosthesis comprises polymeric material;
pressurizing the interior of the pressure vessel to a predetermined pressure;
supplying a mixture of a carrier fluid and a pharmacological agent into the pressure vessel;
exposing the polymeric material and the mixture of carrier fluid and pharmacological agent in the pressure vessel for a time sufficient to swell the polymeric material such that the carrier fluid and pharmacological agent at least partially penetrate the swollen polymeric material; and
releasing the pressure in the pressure vessel such that the carrier fluid diffuses out of the swollen polymeric material and such that a predetermined amount of the pharmacological agent remains elutably trapped within the polymeric material.

32. The method of claim 31, wherein the carrier fluid is carbon dioxide, and wherein the pharmacological agent is hydrophobic.

33. The method of claim 32, wherein the pharmacological agent is everolimus.

34. The method of claim 31, wherein the carrier fluid is water, and wherein the pharmacological agent is hydrophilic.

35. The method of claim 31, wherein pressurizing the interior of the pressure vessel comprises pressurizing the interior of the pressure vessel with carbon dioxide.

36. The method of claim 31, wherein the carbon dioxide is in a supercritical state.

37. The method of claim 36, wherein the carbon dioxide contains one or more of a co-solvent, a surfactant, and a co-surfactant.

38. The method of claim 31, wherein the carrier fluid is configured to alter diffusion coefficients of the polymeric material.

39. The method of claim 37, wherein the co-solvent is selected from the group consisting of ethanol and methanol.

40. The method of claim 31, wherein the intraluminal prosthesis is a stent.

41. The method of claim 31, wherein the polymeric material is erodible.

42. The method of claim 31, wherein the polymeric material is non-erodible.

43. The method of claim 31, wherein the polymeric material is a coating on a portion of the intraluminal prosthesis.

44. The method of claim 41, wherein the erodible polymeric material is selected from the group consisting of, surgical gut, silk, cotton, liposomes, poly(hydroxybutyrate), polycarbonate, polyacrylate, polyanhydride, polyethylene glycol, poly(ortho esters), poly(phosphoesters), polyesters, polyamides, polyphosphazenes, poly(p-dioxane), poly(amino acid), polyglactin, erodible hydrogels, collagen, chitosan, poly(lactic acid), poly(L-lactic acid), poly(D,L-lactic acid), poly(glycolic acid), poly(D-lactic-co-glycolic acid), poly(L-lactic-co-glycolic acid), poly (D,L-lactic-co-glycolic acid), poly(ε-caprolactone), poly(valerolactone), poly(hydroxy butyrate), poly(hydrovalerate), polydioxanone, poly(propylene fumarate), poly(ethyleneoxide)-poly(butylenetetraphthalate), poly(lactic acid-co-lysine), poly(L-lactic acid) and poly(ε-caprolactone) copolymers.

45. The method of claim 31, further comprising:
immersing the intraluminal prosthesis in a mixture of a carrier fluid and radiopaque material; and
pressurizing the mixture of carrier fluid and radiopaque material for a time sufficient to cause the polymeric material to swell such that the carrier fluid and radiopaque material at least partially penetrate the swollen polymeric material.

46. A method of impregnating an intraluminal prosthesis with a pharmacological agent, comprising:
exposing polymeric material of an intraluminal prosthesis to carbon dioxide under conditions sufficient to tackify the polymeric material;
applying a pharmacological agent in micronized, dry form to the tackified polymeric material; and
applying a membrane layer to the intraluminal prosthesis, wherein the membrane layer is configured to allow the pharmacological agent to elute therethrough when the intraluminal prosthesis is deployed within a body of a subject.

47. The method of claim 46, wherein only selected portions of the polymeric material of the intraluminal prosthesis are exposed to carbon dioxide and become tackified.

48. The method of claim 46, wherein the intraluminal prosthesis is masked so as to limit exposure of the base layer to carbon dioxide to only selected portions of the intraluminal prosthesis.

49. The method of claim 46, wherein a plurality of pharmacological agents are applied to the tackified polymeric material.

50. The method of claim 49, wherein the plurality of pharmacological agents comprises a uniform mixture.

51. The method of claim 46, wherein the pharmacological agent is applied by rolling the intraluminal prosthesis in a mass of the pharmacological agent.

52. The method of claim 46, wherein the pharmacological agent is applied by blowing the dry, micronized particles onto the intraluminal prosthesis.

53. The method of claim 46, wherein the membrane layer comprises ethylene vinyl acetate.

54. The method of claim 46, wherein the membrane layer comprises polyethylene glycol.

55. The method of claim 46, wherein the membrane layer comprises a fluoropolymer film.

56. The method of claim 46, wherein the pharmacological agent comprises an antineoplastics.

57. The method of claim 56, wherein the pharmacological agent comprises Paclitaxel.

58. A method of impregnating an intraluminal prosthesis with multiple pharmacological agents, comprising:
exposing polymeric material of an intraluminal prosthesis to carbon dioxide under conditions sufficient to tackify multiple portions of the polymeric material;
applying a respective different pharmacological agent in micronized, dry form to each respective tackified portion of the polymeric material; and
applying a membrane layer to the intraluminal prosthesis, wherein the membrane layer is configured to allow the pharmacological agents to elute therethrough when the intraluminal prosthesis is deployed within a body of a subject.

59. A method of impregnating an intraluminal prosthesis with multiple pharmacological agents, comprising:
exposing polymeric material of an intraluminal prosthesis to carbon dioxide under conditions sufficient to tackify a portion of the polymeric material;
applying a first pharmacological agent in micronized, dry form to the tackified portion of the polymeric material;
applying a first membrane layer to the intraluminal prosthesis, wherein the first membrane layer is configured to allow the first pharmacological agent to elute therethrough when the intraluminal prosthesis is deployed within a body of a subject;
applying a second pharmacological agent to the first membrane layer; and
applying a second membrane layer to the intraluminal prosthesis such that the second pharmacological agent is sandwiched between the first and second membrane layers, and wherein the second membrane layer is configured to allow the second pharmacological agent to elute therethrough when the intraluminal prosthesis is deployed within a body of a subject.

60. An intraluminal prosthesis, comprising:
a tubular body portion comprising polymeric material;
a pharmacological agent in dry, micronized form attached directly to the tubular body portion; and
a membrane attached to the tubular body portion, wherein the membrane overlies the pharmacological agent, wherein the membrane is configured to allow the pharmacological agent to elute therethrough when the intraluminal prosthesis is deployed within a body of a subject.

61. The intraluminal prosthesis of claim 60, wherein the membrane is configured to allow the pharmacological agent to elute at a predetermined rate.

62. The intraluminal prosthesis of claim 60, wherein the tubular body portion comprises an organic-based, erodible material.

63. The intraluminal prosthesis of claim 60, wherein the pharmacological agent is attached directly to the tubular body portion in only selected locations.

64. The intraluminal prosthesis of claim 60, wherein a plurality of pharmacological agents are attached directly to the tubular body portion.

65. The intraluminal prosthesis of claim 64, wherein the plurality of pharmacological agents are homogeneously distributed on the tubular body portion.

66. The intraluminal prosthesis of claim 64, wherein the plurality of pharmacological agents are heterogeneously distributed on the tubular body portion.

67. The intraluminal prosthesis of claim 60, wherein the membrane comprises ethylene vinyl acetate.

68. The intraluminal prosthesis of claim 60, wherein the membrane layer comprises polyethylene glycol.

69. The intraluminal prosthesis of claim 60, wherein the membrane comprises a fluoropolymer film.

70. The intraluminal prosthesis of claim 60, wherein the tubular body portion comprises a first end, a second end, and a flow passage defined therethrough from the first end to the second end, wherein the body portion is sized for intraluminal placement within a subject passage, and wherein the body portion is expandable from a first, reduced cross-sectional dimension to a second enlarged cross-sectional dimension so that the body portion can be transported intraluminally to a targeted portion of a passage and then expanded to the second enlarged cross-sectional dimension so as to engage and support the targeted portion of the passage.

71. The intraluminal prosthesis of claim 60, wherein the intraluminal prosthesis comprises a stent.
